(19) United States
(12) Patent Application Publication
(10) Pub. No.: US 2019/0159713 A1
(43) Pub. Date: May 30, 2019

(54) SYSTEM AND METHOD FOR EVALUATING MENTAL DISEASE TREATMENT CONTENTS USING COMPLEX BIOMETRIC INFORMATION

(71) Applicant: Blaubit Co., Ltd., Seongnam-si (KR)

(72) Inventor: Euisun KIM, Seoul (KR)

(73) Assignee: Blaubit Co., Ltd., Seongnam-si (KR)

(21) Appl. No.: 15/950,382

(22) Filed: Apr. 11, 2018

Related U.S. Application Data
(60) Provisional application No. 62/591,477, filed on Nov. 28, 2017.

(51) Int. Cl. A61B 5/16; A61B 5/04; A61B 5/0205; A61B 5/024; A61B 5/01

(52) U.S. Cl. CPC A61B 5/165; A61B 5/04012; A61B 5/162; A61B 5/0245; A61B 5/02438; A61B 5/01; A61B 5/0205

(57) ABSTRACT

Disclosed herein are a system and a method for evaluating mental disease treatment contents using complex biometric information, which can provide contents of good quality for recovery and treatment of a psychiatric patient, can develop customized treatment contents, and provide objective and logical evaluation information through the treatment contents.

### CROSS-REFERENCE TO RELATED APPLICATION(S)

This application claims the benefit under 35 USC 119(e) of U.S. Provisional Application No. 62/591,477 filed on Nov. 28, 2017, the entire disclosure of which is incorporated herein by reference for all purposes.

### BACKGROUND OF THE INVENTION

### Field of the Invention

The present invention relates to a system and a method for evaluating mental disease treatment contents using complex biometric information, and more particularly, to a system and a method for evaluating mental disease treatment contents using complex biometric information, which can provide contents of good quality for recovery and treatment of a psychiatric patient, can develop customized treatment contents, and provide objective and logical evaluation information through the treatment contents.

### Background Art

People in the modern society have depression, manic-depressive disorder, panic disorder, and so on unwittingly, and most of them are caused by various kinds of stress and individual life environment.

Recently, there are instances where some of the people do a drastic choice, such as suicide or murder.

Reckless driving, driving by revenge, and dating violence, which increase recently, may be examples that are expressed in a bad direction by such patients, who are hiding such mental diseases and keep the diseases to themselves.

Such mental diseases are closely connected not only with individual life but also with family, work, relatives, and surrounding people, and psychologic stress causes lots of emotional illness, such as suicide, seclusive odd one out, interpersonal conflicts, depression, nervousness, and so on.

If such mental diseases are not managed properly, it may deteriorate the quality of individual life and cause nationally serious problems, such as waste of resources.

Various kinds of stress build up, and if there is no proper management of stress, the patients may suffer from difficulty in interpersonal relationship and may reach cut-off of interactions with other people and extreme situations due to worsening in relations.

Because of maladjustment in relations, such persons deny properness of relations and may have pathological diseases, such as depression, emotional disorder, extreme anxiety disorder, self-injurious behaviors, suicide, and so on, due to a sense of loss and a lot of injuries.

Korean Patent No. 10-1534328 discloses a mental health examination device and a mental health management device for a mental health management system. The mental health management system supports a user to manage his or her mental health condition including stress and depression using a user terminal, such as a mobile terminal equipment, without visiting a hospital (mental health hospital) or a mental health clinic. Korean Patent No. 10-1789970 discloses a method for diagnosing and treating mental health online. The method for diagnosing and treating mental health online supports a user to be diagnosed and treated remotely through online in relation with his or her wanted mental health items without visiting a hospital (mental health hospital) or a mental health clinic.

However, such treatment methods carry out diagnosis and treatment using contents of language, images and so on, but have a disadvantage in that it is difficult to provide objective verification of treatment effects using such contents information.

### PATENT LITERATURE

### Patent Documents

Patent Document 1: Korean Patent Publication No. 10-1534328 entitled “mental health examination device and a mental health management device for a mental health management system”

Patent Document 2: Korean Patent Publication No. 10-1789970 entitled “method for diagnosing and treating mental health online”

### SUMMARY OF THE INVENTION

Accordingly, the present invention has been made to solve the above-mentioned problems occurring in the prior arts, and it is an object of the present invention to provide a system and a method for evaluating mental disease treatment contents using complex biometric information, which can provide contents of good quality for recovery and treatment of a psychiatric patient, can develop customized treatment contents, and provide objective and logical evaluation information through the treatment contents.

To accomplish the above object, according to the present invention, there is provided a system for evaluating mental disease treatment contents using complex biometric information including: a sensor unit which is worn on the body of a patient to detect at least one body reaction information; a sensor control unit for converting and outputting the body reaction information detected by the sensor unit into a preset communication format; an information managing unit, which receives the body reaction information outputted from the sensor control unit, transfers it to an information analyzing unit, and outputs virtual reality (VR) contents information for treatment of a mental disease transferred from the information analyzing unit to a display unit; the information analyzing unit, which outputs the present VR contents information to the information managing unit or the display unit, receives the body reaction information transferred from the information managing unit and analyzes the body reaction information by individual body reaction data, and compares the analyzed body reaction data with a preset reference value to create at least one contents evaluation information; and the display unit for displaying the VR contents information outputted from the information managing unit or the information analyzing unit so that the patient can see the VR contents information.

Moreover, the information analyzing unit includes: a contents managing unit, which searches the VR contents information for treatment of the mental disease from a database, and reproduces the VR contents information through the display unit; a contents evaluating unit, which receives the patient's body reaction information transferred from the information managing unit and analyzes the body reaction information by individual body reaction data, and compares the analyzed body reaction data with a preset contents evaluation reference value to create contents evaluation information; and the database, which stores the VR contents information, the patient's body reaction information responding to the VR contents information, and the evaluation information.

Furthermore, the contents evaluating unit classifies and normalizes the created contents evaluation information by at least one of the sympathy index, the cognition index, the emotion index, and the sentiment index, and visualizes the classified indexes so as to display treatment effects by the contents.

Additionally, in another aspect of the present invention, the present invention provides a method for evaluating mental disease treatment contents using complex biometric information including the steps of: a) when a patient's mental disease information is inputted, searching VR contents information for treatment of a mental disease in response to the inputted mental disease information by an information analyzing unit; b) receiving and storing the patient's body reaction information before reproduction of the VR contents information by the information analyzing unit, and displaying the searched VR contents information through a display unit so that the patient can see the VR contents information; c) detecting the patient's body reaction information by a sensor unit, and inputting the detected body reaction information to the information analyzing unit through an information managing unit by a sensor control unit; d) when the information analyzing unit receives the body reaction information transferred from the information managing unit, analyzing the body reaction information by individual body reaction data and normalizing the analyzed body reaction data; and e) comparing the analyzed body reaction data with a preset contents evaluation reference value, creating body reaction data before and after application of contents, and creating at least one contents evaluation information according to the created body reaction data.

In addition, the contents evaluation information classifies and normalizes the body reaction data before and after application of the contents by at least one of the sympathy index, the cognition index, the emotion index, and the sentiment index, and visualizes the classified indexes so as to display treatment effects by the contents.

According to the present invention, the system and method for evaluating mental disease treatment contents using complex biometric information can select and provide contents of good quality for recovery and treatment of a psychiatric patient.

Moreover, the system and method for evaluating mental disease treatment contents using complex biometric information can develop customized treatment contents by individual using the patient's complex body reaction information.

Furthermore, the system and method for evaluating mental disease treatment contents using complex biometric information can provide objective indexes, such as an emotion index, a cognition index, a sympathy index, a sentiment index, and so on, through evaluation of treatment contents.

Additionally, the system and method for evaluating mental disease treatment contents using complex biometric information can provide objective and logical evaluation information by visualizing reaction indexes before and after treatment through the evaluation information of the treatment contents.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a block diagram showing configuration of an evaluation system of mental disease treatment contents using complex biometric information according to a preferred embodiment of the present invention;

FIG. 2 is an exemplary view showing a used state of the evaluation system of mental disease treatment contents using complex biometric information;

FIG. 3 is a block diagram showing a complex sensor unit of the evaluation system of mental disease treatment contents using complex biometric information;

FIG. 4 is a block diagram showing a sensor control unit of the evaluation system of mental disease treatment contents using complex biometric information;

FIG. 5 is an exemplary view showing an operational state of an information managing unit of the evaluation system of mental disease treatment contents using complex biometric information;

FIG. 6 is a block diagram showing an information analyzing unit of the evaluation system of mental disease treatment contents using complex biometric information;

FIG. 7 is an exemplary view showing comparison of waveforms measured before and after treatment by a method for evaluating mental disease treatment contents using complex biometric information according to a preferred embodiment of the present invention; and

FIG. 8 is a flow chart showing the method for evaluating mental disease treatment contents using complex biometric information.

### DETAILED DESCRIPTION OF THE PREFERRED EMBODIMENT

Hereinafter, reference will be now made in detail to the preferred embodiments of the present invention with reference to the attached drawings.

In this description, when a part “includes” or “comprises” an element, the part can further include other elements, not excluding the other elements.

Moreover, terms such as “part”, “unit” and “module” indicate a unit for processing at least one function or operation, wherein the unit and the block may be embodied as hardware or software or embodied by combining hardware and software.

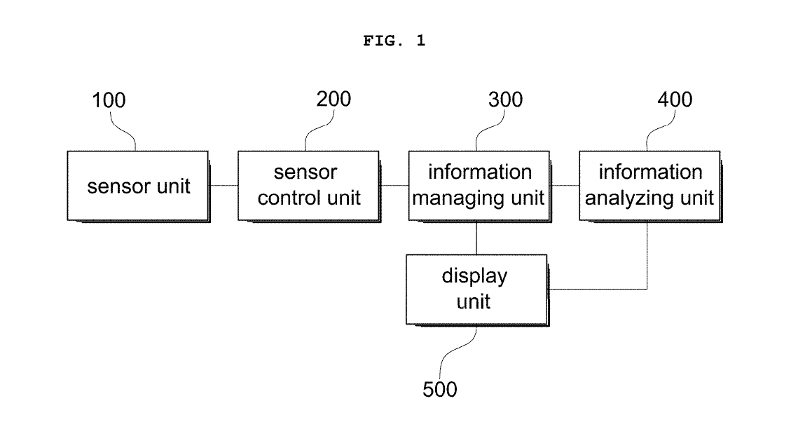

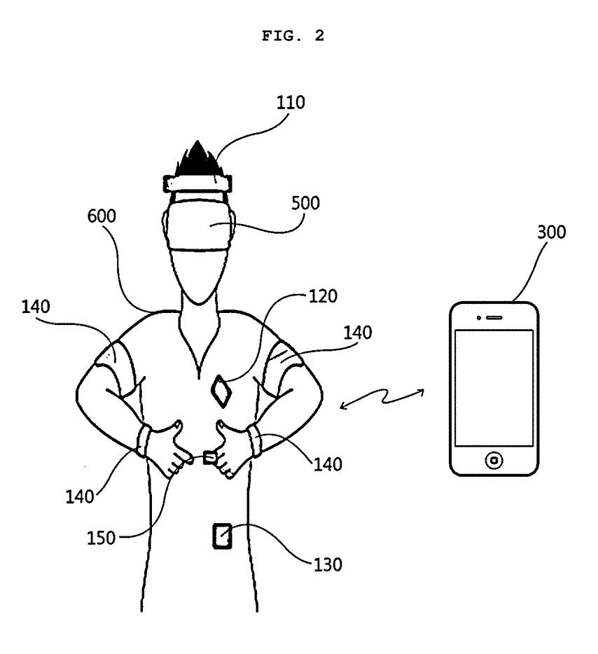

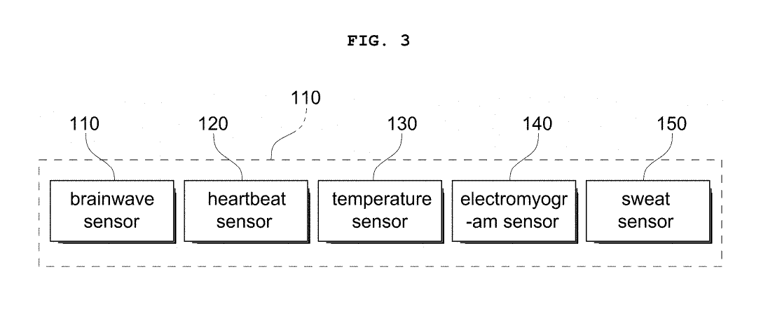

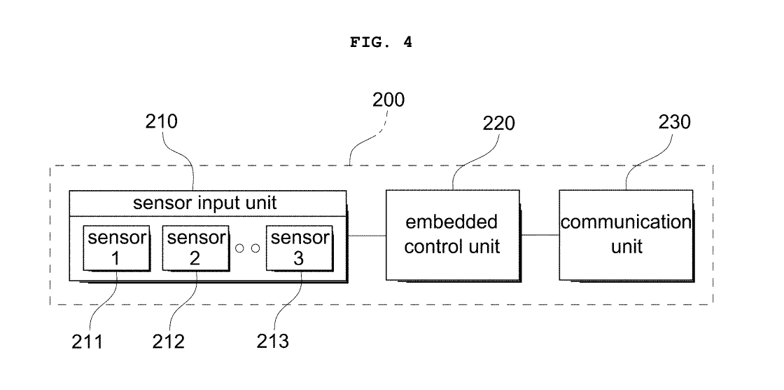

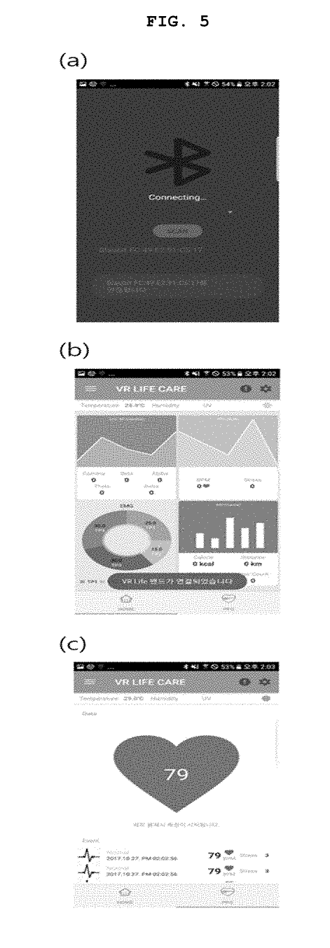

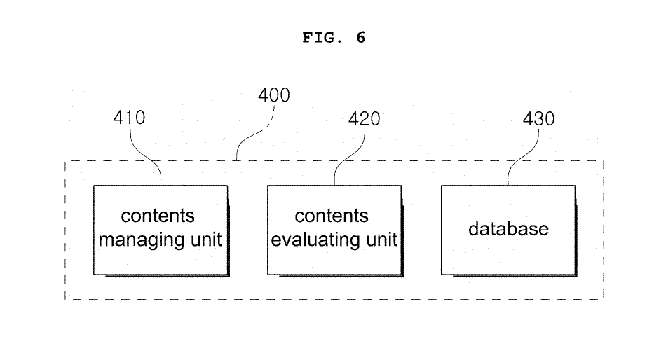

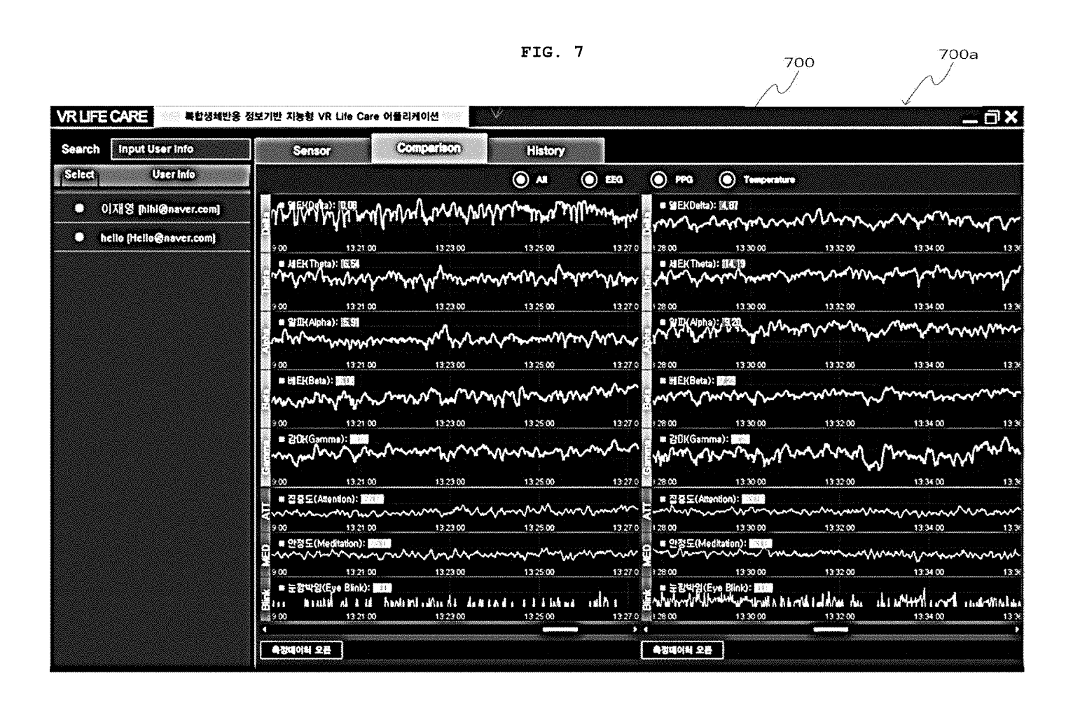

FIG. 1 is a block diagram showing configuration of an evaluation system of mental disease treatment contents using complex biometric information according to a preferred embodiment of the present invention, FIG. 2 is an exemplary view showing a used state of the evaluation system of mental disease treatment contents using complex biometric information, FIG. 3 is a block diagram showing a complex sensor unit of the evaluation system of mental disease treatment contents using complex biometric information, FIG. 4 is a block diagram showing a sensor control unit of the evaluation system of mental disease treatment contents using complex biometric information, FIG. 5 is an exemplary view showing an operational state of an information managing unit of the evaluation system of mental disease treatment contents using complex biometric information, FIG. 6 is a block diagram showing an information analyzing unit of the evaluation system of mental disease treatment contents using complex biometric information, and FIG. 7 is an exemplary view showing comparison of waveforms measured before and after treatment by a method for evaluating mental disease treatment contents using complex biometric information according to a preferred embodiment of the present invention.

As shown in FIGS. 1 to 7, the evaluation system of mental disease treatment contents using complex biometric information includes a sensor unit 100, a sensor control unit 200, an information managing unit 300, an Information analyzing unit 400, and a display unit 500.

The sensor unit 100 is worn on the body of a patient 600 to detect at least one body reaction information. The sensor unit 100 includes: a brainwave sensor 110 worn on the patient's head to measure brainwave signals, such as delta, theta, alpha, SMR, low beta, high beta, gamma, and so on; a heartbeat sensor 120 for measuring a cardiac rate of the patient 600; a temperature sensor 130 for measuring body temperature of the patient 600; an electromyogram sensor 140 for measuring skeletal muscle action current of the patient 600; and a sweat sensor 150 for measuring the patient's secretions, such as sweat.

For convenience in description, in this embodiment, the sensor unit 100 has five sensors for measuring the patient's body reaction information, but the present invention is not limited to the above, and it is obvious by those of ordinary skill in the art that the sensor unit 100 may be formed by combination of a gyro sensor and a motion tracking sensor for detecting body movements, and various sensors for measuring the patient's body conditions.

The sensor control unit 200 is to convert and output the body reaction information detected from the sensor unit 100 into a preset communication format. The sensor control unit 200 includes a sensor input unit 210 electrically getting in contact with the sensor unit 100, an embedded control unit 220 for controlling operation of the sensor control unit 200, and a communication unit 230.

The sensor input unit 210 has a plurality of connection terminals including input terminals of a first sensor 211, a second sensor 212, . . . and a n^{th} sensor 213 so that the sensors are connected with one another.

The embedded control unit 220 is connected with the information managing unit 300 through a wire or wireless communication network so that body reaction information detected by the sensor input part 210 can be transferred to the information managing unit 300.

The communication unit 230 converts the body reaction information detected through the sensor input unit 210 into a preset communication format signal and outputs the converted signal to the information managing unit 300, and in this instance, the measured body reaction information may be transferred through various communication formats, such as wire communication using an USB, Zigbee, Bluetooth, low power wireless communication, RF communication, and so on.

The information managing unit 300 receives the body reaction information outputted from the sensor control unit 200 and transfers it to the information analyzing unit 400.

Moreover, the information managing unit 300 is configured to be connected with the display unit 500, and receives virtual reality (VR) contents information to treat a mental disease, which is transferred from the information analyzing unit 400, and outputs the VR contents information to the display unit 500. Preferably, application program may be installed by a portable tablet PC, a smart phone or the like.

That is, the information managing unit 300 transfers and receives data to and from the sensor control unit 200, and receives the VR contents information transferred from the information analyzing unit 400 such that application program is installed to display the VR contents information on the display unit 500.

Furthermore, the application program detects connection information of the information analyzing unit 400 and is connected with the information analyzing unit 400 through a network, so that the body reaction information detected from the sensor unit 100 by the information managing unit 300 is transferred to the information analyzing unit 400 and the detected body reaction information may be directly displayed through the information managing unit 300.

That is, the operation for checking the connection status with the sensor control unit 200 as shown in FIG. 5A and operational statuses of the sensors detected from the sensor control unit 200 as shown in FIG. 5B are displayed, or an operational status of a specific sensor according to the patient's selection as shown in FIG. 5C is displayed.

Additionally, the information managing unit 300 detects the patient's body reaction information and transfers it to the information analyzing unit 400 before the VR contents information is reproduced.

The information analyzing unit 400 outputs the VR contents information to the information managing unit 300 or the display unit 500, and receives the body reaction information transferred from the information managing unit 300 to analyze it by individual body reaction data. After that, the information analyzing unit 400 compares the analyzed body reaction data with a preset reference value for evaluation in order to create at least one contents evaluation information. The information analyzing unit 400 may be a desktop PC, a notebook PC, a server, or the like having a contents managing unit 410, a contents evaluating unit 420, and a database 430, and preferably, may be a server.

The contents managing unit 410 searches the VR contents information for treating mental diseases from the database 430, and reproduces the VR contents information through the display unit 500.

The contents evaluating unit 420 receives the patient's body reaction information measured by the sensor unit 100 in relation with the VR contents information reproduced through the display unit 500 and analyzes the body reaction information by individual body reaction data. After that, the contents evaluating unit 420 compares the analyzed body reaction data with a preset reference value for evaluation to create contents evaluation information.

Moreover, the contents evaluating unit 420 classifies and normalizes the created contents evaluation information by at least one of a sympathy index, a cognition index, an emotion index, and a sentiment index, so as to make multilateral analysis possible. The classified indexes are visualized so that treatment effects by the contents are displayed.

That is, the contents evaluating unit 420 analyzes the patient's body reaction information relative to the VR contents information transferred from the information managing unit 300 into body reaction data by each sensor, and normalizes the analyzed body reaction data according to previously established regulations so as to create object and logical indexes.

Furthermore, the contents evaluating unit 420 compares the normalized and analyzed body reaction data with the preset reference value for evaluation of the contents in order to judge treatment effects, and then, as shown in FIG. 7, creates comparison data of a measurement waveform 700 before application of the contents to the patient's body reaction with a measurement waveform 700a after application of the contents, and creates contents evaluation information relative to treatment effects based on the created body reaction comparison data.

The VR contents information contains various kinds of information, such as sounds, images, waveforms, and so on and various kinds of information for treatment of the patient.

The database 430 stores the VR contents information, the patient's body reaction information responding to the VR contents information, and evaluation information of the contents for evaluation.

The display unit 500 is connected with the information managing unit 300 or the information analyzing unit 400 to display the VR contents information outputted from the information managing unit 300 or the information analyzing unit 400 so that the patient 600 can see the VR contents information. The display unit 500 may be one of a monitor or a head mount display (HMD) worn on the patient's head, preferably, the head mount display.

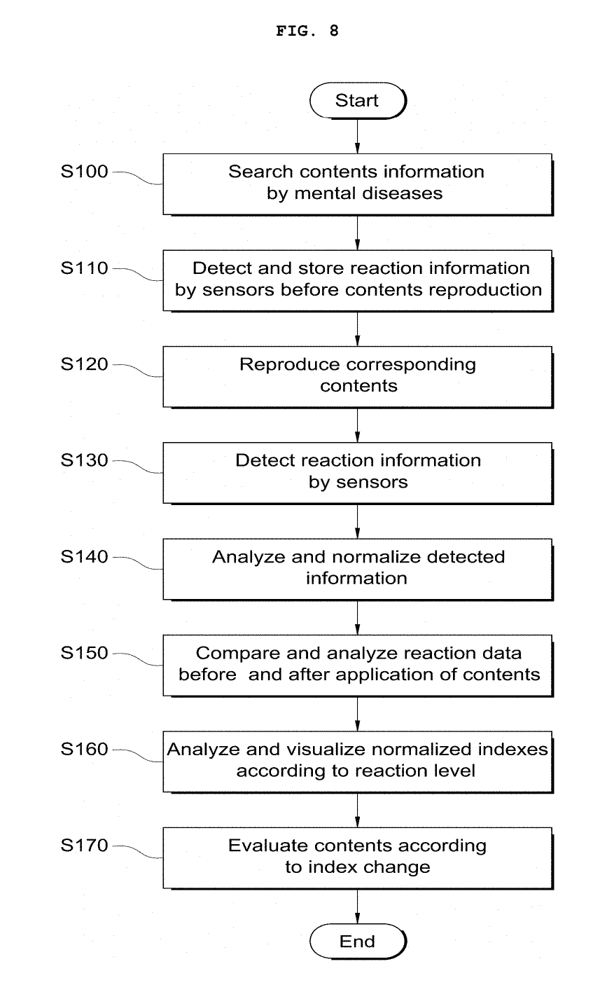

Next, referring to FIGS. 1 to 8, a method for evaluating mental disease treatment contents using complex biometric information according to a preferred embodiment of the present invention will be described.

First, when a plurality of the sensor units 100 are mounted on the patient's body, the sensor control unit 200 is connected with the information managing unit 300 and notifies the patient that measurement of the patient's body reaction information is in ready status, and the information managing unit 300 is connected with the information analyzing unit 400 through wire or wireless network to prepare for measurement.

After preparation for measurement is completed, when the patient's mental disease information is inputted through the information managing unit 300 or the information analyzing unit 400, the information analyzing unit 400 searches VR contents information for mental disease treatment, which responds to the inputted mental disease information (S100).

The information analyzing unit 400 receives and stores the patient's body reaction information before reproduction of the VR contents information. When the patient's initial body reaction information is stored completely, the VR contents information searched in the step S100 is displayed through the display unit 500 (S120).

In the step S120, when the display unit 500 is connected with the information analyzing unit 400, the information analyzing unit 400 controls the VR contents information to be displayed through the display unit 500. When the display unit 500 is connected with the information managing unit 300, the information analyzing unit 400 transfers the VR contents information to the information managing unit 300 so that the information managing unit 300 controls the VR contents information to be displayed through the display unit 500.

If the display unit 500 is the head mount display, the display unit 500 may be connected with the information managing unit 300 for portability and convenience in use.

When the VR contents information is displayed in the step S120, the sensor control unit 200 detects the patient's body reaction information from the sensor units 100 worn on the patient's body (S130), and the detected body reaction information is inputted to the information analyzing unit 400 through the information managing unit 300.

In the step S130, the information managing unit 300 displays the patient's body reaction information transferred from the sensor control unit 200 through displaying means as shown in FIGS. 5B and 5C.

When the information analyzing unit 400 receives body reaction information transferred from the information managing unit 300, the information analyzing unit 400 analyzes the received body reaction information by individual body reaction data, and normalizes the analyzed body reaction data according to the preset reference value (S140).

In the step S140, the normalized body reaction data compares and analyzes the body reaction data analyzed by the information analyzing unit 400 with the present contents evaluation reference value (S150), and then, creates contents evaluation information according to body reactions before and after application of the contents (S160).

That is, the information analyzing unit 400 classifies and normalizes the body reaction data before and after application of the contents by at least one of the sympathy index, the cognition index, the emotion index, and the sentiment index, and then, visualizes the classified indexes so as to create contents evaluation information to display treatment effects by the contents.

The information managing unit 400 outputs results of the VR contents information on the basis of the contents evaluation information created in the step S160.

Therefore, the system and method for evaluating mental disease treatment contents using complex biometric information can select and provide contents of good quality for recovery and treatment of a psychiatric patient.

Moreover, the system and method for evaluating mental disease treatment contents using complex biometric information can develop customized treatment contents by individual using the patient's complex body reaction information, which patents show in different ways by individual in connection with different symptoms.

Furthermore, the system and method for evaluating mental disease treatment contents using complex biometric information can provide objective indexes, such as an emotion index, a cognition index, a sympathy index, a sentiment index, and so on, through evaluation of treatment contents.

Additionally, the system and method for evaluating mental disease treatment contents using complex biometric information can provide objective and logical evaluation information by visualizing reaction indexes before and after treatment through the evaluation information of the treatment contents.

As described above, while the present invention has been particularly shown and described with reference to the example embodiments thereof, it will be understood by those of ordinary skill in the art that various changes, modifications and equivalents may be made therein without departing from the idea and scope of the present invention described in the following claims.

Additionally, the reference numerals described in the claims are written for clarity and convenience in description, and the present invention is not limited to the reference numerals. Moreover, in the drawings, thicknesses of lines and sizes of constituent elements may be exaggerated for clarity and convenience in explanation. Further, wordings to be described later are defined in consideration of the functions of the present invention, and may differ depending on the intentions of a user or an operator or custom. Accordingly, such wordings should be defined on the basis of the contents of the overall specification.

What is claimed is:

1. A system for evaluating mental disease treatment contents using complex biometric information comprising:
a sensor unit (100) which is worn on the body of a patient (600) to detect at least one body reaction information;
a sensor control unit (200) for converting and outputting the body reaction information detected by the sensor unit (100) into a preset communication format;
an information managing unit (300), which receives the body reaction information outputted from the sensor control unit (200), transfers it to an information analyzing unit (400), and outputs virtual reality (VR) contents information for treatment of a mental disease transferred from the information analyzing unit (400) to a display unit (500);
the information analyzing unit (400), which outputs the present VR contents information to the information managing unit (300) or the display unit (500), receives the body reaction information transferred from the information managing unit (300) and analyzes the body reaction information by individual body reaction data, and compares the analyzed body reaction data with a preset reference value to create at least one contents evaluation information; and
the display unit (500) for displaying the VR contents information outputted from the information managing unit (300) or the information analyzing unit (400) so that the patient (600) can see the VR contents information.

2. The evaluation system according to claim 1, wherein the information analyzing unit (400) comprises:
a contents managing unit (410), which searches the VR contents information for treatment of the mental disease from a database (430), and reproduces the VR contents information through the display unit (500);
a contents evaluating unit (420), which receives the patient's body reaction information transferred from the information managing unit (300) and analyzes the body reaction information by individual body reaction data, and compares the analyzed body reaction data with a preset contents evaluation reference value to create contents evaluation information; and
the database (430), which stores the VR contents information, the patient's body reaction information responding to the VR contents information, and the evaluation information.

3. The evaluation system according to claim 2, wherein the contents evaluating unit (420) classifies and normalizes the created contents evaluation information by at least one of the sympathy index, the cognition index, the emotion index, and the sentiment index, and visualizes the classified indexes so as to display treatment effects by the contents.

4. A method for evaluating mental disease treatment contents using complex biometric information comprising the steps of:
a) when a patient's mental disease information is inputted, searching VR contents information for treatment of a mental disease in response to the inputted mental disease information by an information analyzing unit (400);
b) receiving and storing the patient's body reaction information before reproduction of the VR contents information by the information analyzing unit (400), and displaying the searched VR contents information through a display unit (500) so that the patient (600) can see the VR contents information;
c) detecting the patient's body reaction information by a sensor unit (100), and inputting the detected body reaction information to the information analyzing unit (400) through an information managing unit (300) by a sensor control unit (200);
d) when the information analyzing unit (400) receives the body reaction information transferred from the information managing unit (300), analyzing the body reaction information by individual body reaction data and normalizing the analyzed body reaction data; and
e) comparing the analyzed body reaction data with a preset contents evaluation reference value, creating body reaction data before and after application of contents, and creating at least one contents evaluation information according to the created body reaction data.

5. The evaluation method according to claim 4, wherein the contents evaluation information classifies and normalizes the body reaction data before and after application of the contents by at least one of the sympathy index, the cognition index, the emotion index, and the sentiment index, and visualizes the classified indexes so as to display treatment effects by the contents.
